(19) United States
(12) Patent Application Publication
(10) Pub. No.: US 2016/0168542 A1
(43) Pub. Date: Jun. 16, 2016

(54) TISSUE ENGINEERED MODELS OF CANCERS

(71) Applicant: THE TRUSTEES OF COLUMBIA UNICERSITY IN THE CITY OF NEW YORK, New York, NY (US)

(72) Inventors: Aranzazu Villasante, New York, NY (US); Gordana Vunjak-Novakovic, New York, NY (US)

(73) Assignee: THE TRUSTEES OF COLUMBIA UNICERSITY IN THE CITY OF NEW YORK, New York, NY (US)

(21) Appl. No.: 14/908,870

(22) PCT Filed: Aug. 1, 2014
(86) PCT No.: PCT/US14/49416

§ 371 Date: Jan. 29, 2016

Related U.S. Application Data
(60) Provisional application No. 61/861,957, filed on Aug. 2, 2013.
(60) Provisional application No. 61/862,447, filed on Aug. 5, 2013.

(51) Int. Cl. C12N 5/09; C12M 1/00; C12M 1/12; B01J 19/00; C12M 3/00

(52) U.S. Cl. CPC C12N 5/0693; B01J 19/0046; C12M 21/08; C12M 25/14; C12M 29/00; C12N 2533/90; C12N 2503/02; B01J 2219/00743; B01J 2219/00497; B01J 2219/00351

(57) ABSTRACT

A 3D decellularized bone scaffold seeded with cancer cells, such as prostate cancer cells or Ewing's sarcoma is provided. It provides platform technology for controllable, quantitative, long-term studies of tissue-engineered tumors, including prostate cancer and Ewing's sarcoma. The scaffold can be used with cancer cell lines to identify therapeutic targets to slow, stop, and reverse tumor growth and progression as well as to predict the efficacy of potential therapeutics.

### CROSS-REFERENCE TO RELATED APPLICATIONS

This application claims the benefit of U.S. Provisional Application No. 61/861,957, filed Aug. 2, 2013 and claims the benefit of U.S. Provisional Application No. 61/862,447, filed Aug. 5, 2013.

### BACKGROUND OF THE DISCLOSED SUBJECT MATTER

1. Field of the Disclosed Subject Matter

The disclosed subject matter relates to tissue engineered models of cancers, including prostate cancer and Ewing's Sarcoma. Particularly, the presently disclosed subject matter relates to providing a three-dimensional decellularized bone scaffold seeded with cancer cells, such as prostate cancer cells and Ewing's sarcoma cells, and bone tissue cells.

2. Background

Cancer research is experiencing tremendous advances in the development of genome-wide regulatory models and network-based methods that helped discover new cancer genes and new mechanisms of drug action. At the same time, there is a growing notion on how important are the environmental contributors to the initiation, progression and suppression of cancer, including the three-dimensionality, other cells, tissue matrix, molecular and physical signaling. The lack of ability to replicate in vitro the complex in vivo milieu of human cancer is a critical barrier to evaluation of the potential therapeutic targets for clinical application.

Ewing's sarcoma is a rare cancer that typically affects the bones. Most often it is found in the leg and arm bones of children, accounting for 1% of all childhood cancers. Ewing's sarcoma can be treated successfully in 50% to 75% of cases. Ewing's sarcoma is a poorly differentiated tumor of uncertain histogenesis and aggressive biologic behavior characterized by a strong membrane staining for CD99. Most prostate cancer deaths are due to metastasis into bone, and yet there is not a good model of metastatic prostate cancer: in vitro, the cancer cells rapidly lose their cancer phenotype, and in vivo the mouse bone is not permissive for cancer cell invasion.

Current experimental methods and models to study cancer growth and progression mainly utilize in vitro two dimensional (2D) co-culturing of cancer specific cell lines and other cells found local in the tumor. However, these 2D models fail to capture the true three dimensional (3D) progression of tumors and are limited in their ability to identify therapeutic targets. The shortcomings are underscored by the fact that most drugs fail to translate observed in vitro effects to in vivo studies and that only about 5% of drugs show effects in clinical trials. Numerous two-dimensional (2D) culture studies and in vivo studies have been actively pursued to further understand the complex mechanisms and the molecular pathways in prostate cancer and Ewing's sarcoma. However these models are not able to mimic the disease. Cells lose relevant properties in 2D due to the loss of physiological extracellular matrix (ECM) when cultured on artificial plastic surfaces at high serum concentrations. Studies in animal models also have their limitations. Prostate cancer and Ewing's sarcoma are human diseases and that are not accurately represented in an animal model.

Based on studies in genetically engineered mice and using clinical data, it has been established that mouse bone acts as a barrier to prostate cancer cell invasion, in contrast to the human bone that is permissive to metastasis.

Thus, there remains a need for a three dimensional model enabling more accurate modelling of cancers.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To address the challenges noted above, the present subject matter provides an advanced platform technology for controllable, quantitative, long-term studies of tissue-engineered tumors, such as prostate cancer and Ewing sarcoma (ES) as clinically significant models. In accordance with the subject matter, a 3D decellularized bone scaffold seeded with cancer cells, such as prostate cancer cells and Ewing's sarcoma cells (patient derived or cell lines) and bone tissue cells is provided. Some genes up-regulated in primary Ewing's sarcoma cells are silenced in existing Ewing's sarcoma cell lines. Thus, the technology of the present disclosure has demonstrated that cancer cells such as prostate cancer and Ewing's sarcoma cell lines cultured in this 3D scaffold re-express the silenced genes, better recapitulating the original in vivo tumor phenotype. Accordingly, the scaffold can be used with cancer cell lines, such as prostate cancer and Ewing's sarcoma, to identify therapeutic targets to slow, stop, and reverse tumor growth and progression as well as predict the efficacy of potential therapeutics. The technology can also be used with patient-derived cancer cells and mesenchymal stem cells for a personalized approach to cancer treatment.

In one aspect, cancer cells are introduced into bone tissue engineered from human cells, and cultured over long periods of time with vascular perfusion, oxygen control, and mechanical loading. Culturing tumor cells in a living bone environment may recapitulate the original in vivo tumor signature.

In accordance with one method, a tissue-engineered model of Ewing's sarcoma is established. A control of oxygen supply and incorporated perfusable vasculature into the engineered ES model is provided.

In accordance with another method, a validation is provided to validate the model by assessing effects of mechanical stress and perfusion on tumor phenotype and focal adhesion genes.

Further, a validation of the advanced bioengineering platform technology for cancer research, in two modifications: (1) for high-throughput screening (96-well format) and advanced studies of tumor biology (24-well format) is provided. The present technology has an unusually high transformative potential; it enables critical advances in several areas central to cancer research, and uses pioneering approaches with potential for paradigm-shifting advances, and is based on pilot data.

In one embodiment, a cancer model is provided with a biomimetic microenvironment representing the pathophysiology of this malignancy. This is achieved by using three-dimensional (3D) instead of conventional two-dimensional (2D) cultures, with the aid of bone-engineering technology. In a 3D context, cancer cell lines modify their 2D transcriptional profile, recapitulating better the original tumor phenotype. This novel model is expected to be a powerful tool for predictive testing of anti-cancer and anti-metastatic compounds.

In some embodiments, a three-dimensional cancer model is provided. The model includes a decellularized bone scaffold and a plurality of cells arrayed on the scaffold. In some embodiments, the plurality of cells comprises cancer cells. In some embodiments, the cancer cells are metastatic cancer cells, prostate cancer cells, or Ewing's sarcoma cells. In some embodiments, the cancer cells comprise a plurality of spheroids. In some embodiments, the bone scaffold comprises a plurality of perfusion channels. In some embodiments, the plurality of cells comprises stem cells. In some embodiments, the plurality of cells comprises osteoblasts. In some embodiments, the plurality of cells comprises bone tissue cells. In some embodiments, the plurality of cells comprises patient-derived cells. In some embodiments, the scaffold is adapted for insertion in one well of a multiple well plate. In some embodiments, the scaffold is adapted for insertion in one well of a 96-well plate. In some embodiments, the scaffold is adapted for insertion in one well of a 24-well plate. In some embodiments, the scaffold has an outer region, and inner region, and a core region. In some embodiments, a first portion of the plurality of cells is arrayed in the outer region, a second portion of the plurality of cells is arrayed in the inner portion, and a third portion of the plurality of cells is arrayed in the core region. In such embodiments, the second portion is hypoxic and the third portion is necrotic.

In some embodiments, a platform for modelling cancer is provided. The platform includes a decellularized bone scaffold, an oxygen supply in gaseous communication with the bone scaffold, a vasculature in fluid communication with the bone scaffold, and a mechanical load coupled to the bone scaffold. In some embodiments, the mechanical load is adapted to apply a mechanical stress to the bone scaffold. In some embodiments, the vasculature comprises a nutrient supply.

In some embodiments, a bioreactor is provided. The bioreactor includes a decellularized bone scaffold, an oxygen supply in gaseous communication with the bone scaffold and a vasculature in fluid communication with the bone scaffold. In some embodiments, the bioreactor is adapted to provide a biomimetic microenvironment to the scaffold.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1A-C illustrate tissue-engineered models of Ewing's sarcoma (TE-ES) according to embodiments of the present disclosure.

FIGS. 2A-C illustrates characterization of TE-ES models according to embodiments of the present disclosure.

FIGS. 3A-D illustrate expression of hypoxic and glycolytic tumor phenotypes according to embodiments of the present disclosure.

FIGS. 4A-D illustrate angiogenesis and vasculogenic mimicry according to embodiments of the present disclosure.

FIG. 5 illustrates re-expression of tumor genes in a 3D tissue-engineered model of Ewing's sarcoma according to embodiments of the present disclosure.

FIGS. 6A-F illustrate generation and characterization of TE-bone according to embodiments of the present disclosure.

FIGS. 7A-D illustrate characterization of Ewing's sarcoma cell lines according to embodiments of the present disclosure.

FIG. 8 illustrates focal adhesion genes and cancer genes expressed in Ewing's sarcoma tumors and bone but not in cell lines according to embodiments of the present disclosure.

FIG. 9 illustrates focal adhesion genes differentially expressed in Ewing's sarcoma tumors and tumor cell lines according to embodiments of the present disclosure.

FIG. 10 illustrates cancer related genes differentially expressed in Ewing's sarcoma tumors and tumor cell lines according to embodiments of the present disclosure.

FIG. 11 illustrates focal adhesion and cancer genes differentially expressed in Ewing's sarcoma tumors and cell lines according to embodiments of the present disclosure.

FIGS. 12A-C illustrates an NPK mouse model according to embodiments of the present disclosure.

FIG. 13 illustrates the differences between mouse prostate tumors and human bone mets according to embodiments of the present disclosure.

### DETAILED DESCRIPTION OF EXEMPLARY EMBODIMENTS

Reference will now be made in detail to exemplary embodiments of the disclosed subject matter, an example of which is illustrated in the accompanying drawings. Methods and corresponding steps of the disclosed subject matter will be described in conjunction with the detailed description of the system.

Overview

Cell Culture. Human Ewing's sarcoma SK-N-MC and RD-ES cell lines were purchased from American Type Culture Collection (ATCC) and cultured according to the manufacturer's specifications. Ewing's sarcoma tumors were obtained from Columbia University Tissue bank. Human Mesenchymal Stem Cell (hMSC) cultivation, seeding and osteogenic differentiation, were performed.

Pellets formation: To form pellets, 0.3×10⁶ Ewing's sarcoma cells were centrifuged in 15 mL Falcon tubes with 4 mL of medium and cultured at 37° C. with 5% humidified CO₂ for one week. Tissue engineered model of tumor. Scaffolds (4 mm diameter×4 mm high cylinder) were prepared from fully decellularized bone, seeded with 1.5×10⁶ hMSCs (passage 3) and incubated in 6 mL of osteogenic medium for 4 weeks. Medium was changed biweekly. After 4 weeks, the scaffolds were bisected; one half was seeded with Ewing's sarcoma cells (3 pellets per scaffold) and the other half was used as a control.

Microarray data analysis. Expression of genes in Ewing's Sarcoma and cell lines was studied in 11 cell lines and 11 tumors by applying the barcode method. A probe set was considered expressed in cell lines/tumors only if detected in all cell lines/tumors. Where a gene had multiple probe sets, the gene was only counted once. Genes expressed in cell lines, but not tumors, or in tumors, but not cell lines, were identified from the asymmetric difference of both sets.

Quantitative real-time PCR (qRT-PCR). Total RNA was obtained using Trizol (Life Technologies) following the manufacturer's instructions. RNA preparations(2 μg) were treated with “Ready-to-go you-prime first-strand beads” (GE Healthcare) to generate cDNA. Quantitative real-time PCR was performed using DNA Master SYBR Green I mix (Applied Biosystems). mRNA expression levels were quantified applying the ΔCt method, ΔCt=(Ct of gene of interest—Ct of GAPDH). Histology and Immunohistochemistry (IHC). Ewing's sarcoma models were fixed in 10% formalin, embedded in paraffin, sectioned into 4 μm slices and stained with haematoxylin and eosin (H/E). Engineered models and tumor samples were stained for CD99, osteopontin (OPN), bone sialoprotein (BSP), and osteocalcin (OCN).

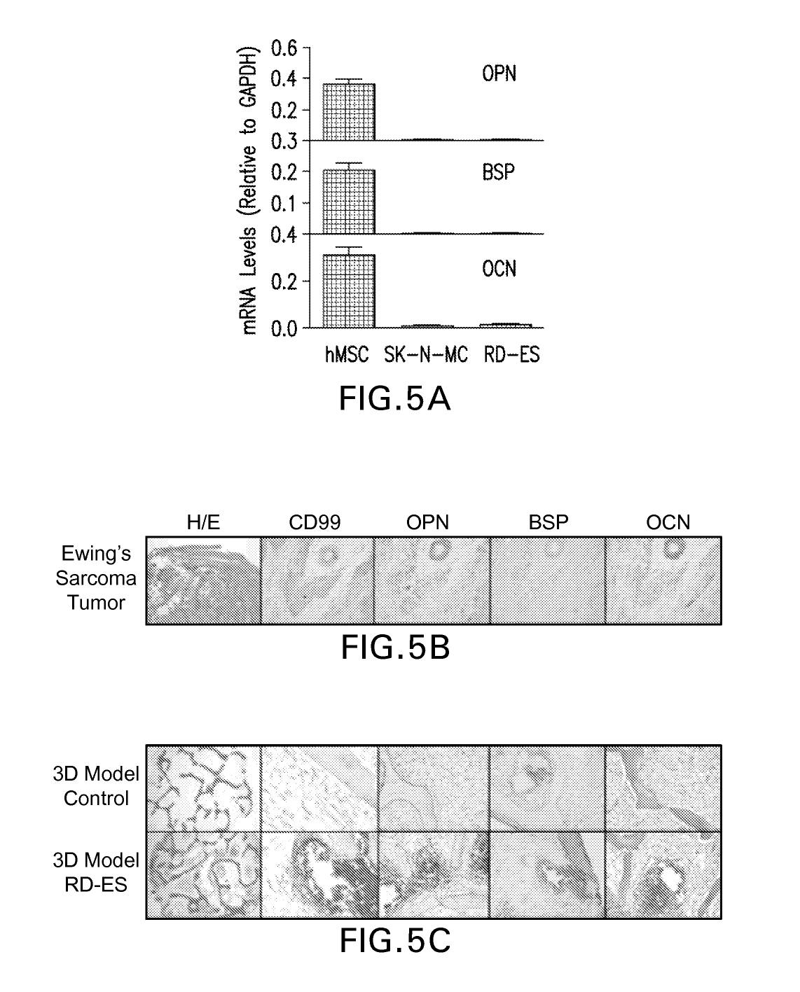

With regard to FIG. 5, re-expression of tumor genes in a 3D tissue-engineerted model of Ewing's sarcoma is depicted. Analysis of Osteopontin (OPN), Bone Sialoprotein (BSP), and Osteocalcin (OCN) in Ewing's sarcoma cell lines cultured in 2D, native tumor samples and engineered 3D model of Ewing's sarcoma (RD-ES). (A) Analysis by qRT-PCR of the indicated ECM genes in two Ewing's sarcoma cell lines and in hMSC. Values correspond to the average±SD (n=3). (B) Immunostains of Ewing's sarcoma tumor samples. (C) Immunostains of the healthy controls and RD-ES engineered bone samples. Representative sections stained for Hematoxylin/Eosin and for CD99 and ECM proteins are shown.

RESULTS: By comparing gene expression profiles of clinical tumor samples and Ewing sarcoma cell lines, genes were identified that were expressed in tumors but not in cell lines. Bioinformatics analysis showed 599 genes up-regulated in tumors and not in the cells. By qRT-PCR 33 genes were identified that were implicated in focal adhesion and cancer. The three MEC proteins (OPN, BSP and OSC) were not expressed in tumor cells (FIG. 5A), in contrast to the actual Ewing's sarcoma tumor samples that expressed high levels of these proteins (FIG. 5B). Notably, when the same tumor cells were cultured within the context of the engineered bone tumor model, they re-expressed all three proteins (FIG. 5C).

In contrast, immortalized Ewing's sarcoma cells cultured in 2D do not express genes implicated in important pathways related to focal adhesion and cancer and expressed at high levels in tumor tissues. An in vitro model of Ewing's sarcoma tumor was constructed by introducing the tumor cells into an engineered bone environment, which showed that the tumor cells re-expressed the silenced genes under these conditions. This Ewing's sarcoma model can serve as a tool for cancer drug discovery and target identification because it provides gene profiles of the tumor cells similar to those in a native tumor.

Tumor Bone-Engineered Model

Cell culture and animal models have tremendously advanced our understanding of cancer biology. However both systems have limitations. Herein is described a bioengineered model of human Ewing's sarcoma that mimics the in vivo bone tumor niche with high biological fidelity. In this model, cancer cells that have lost their transcriptional profiles after monolayer culture re-express genes related to focal adhesion and cancer pathways. The bioengineered model recovers the original hypoxic and glycolytic tumor phenotype, and leads to re-expression of angiogenic and vasculogenic mimicry features that favor tumor adaptation. Differentially expressed genes between the monolayer cell culture and tumor environment are potential therapeutic targets that can be explored using the bioengineered tumor model.

Both the two-dimensional (2D) culture and in vivo models of cancer may be used to unravel the complex mechanisms and molecular pathways of cancer pathogenesis. Cancer cells lose many of their relevant properties in 2D culture, due to the lack of the native-like physiological milieu with 3D extracellular matrix (ECM), the other cells and regulatory factors. As a result, 2D cultures are not predictive of antitumoral drug effects in the human being. Animal models have their own limitations in representing human disease, necessitating the use of clinical data. While simple 3D models of cancer, such as tumor spheroids, cell inserts, and cell encapsulation in hydrogels or porous scaffolds are an advance over monolayer cultures, cancer cells still remain deprived of native tumor environments where cancer cell-nonmalignant cell interactions are crucial for tumor biology. Indeed, the microenvironment can both inhibit and facilitate tumor growth and metastatic dissemination to distant organs. Current approaches are far from replicating the native in vivo milieu in which tumors develop, a necessary condition for advancing cancer research and translating novel therapies into clinical practice.

The present disclosure describes a model of human bone cancer (such as prostate cancer and Ewing's sarcoma) engineered by introducing tumor cell spheroids into their resident bone tissue environment that has been formed by culturing human mesenchymal stem cells in decellularized bone matrix. This model allows not only the cross-talk between the cancer cells, but also the interactions of cancer cells with the human bone cells and the mineralized bone matrix. Within such native-like environment, cancer cells (i) re-express focal adhesion and cancer related genes that are highly expressed in tumors but lost in monolayer cultures, (ii) recapitulate the original hypoxic and glycolytic tumor phenotypes, and (iii) acquire angiogenic capacity and vasculogenic mimicry that favor tumor initiation and adaptation. Bioengineered models of human bone cancer can be valuable tools for identifying genes that are differentially expressed between cell lines and tumors, and thus representing potential therapeutic targets.

Tissue-Engineered Model of Ewing's Sarcoma (TE-ES)

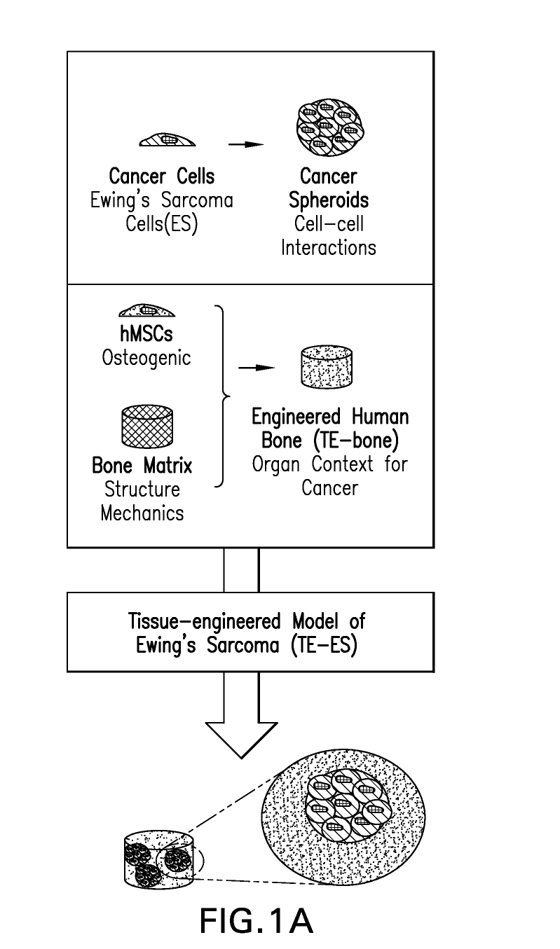

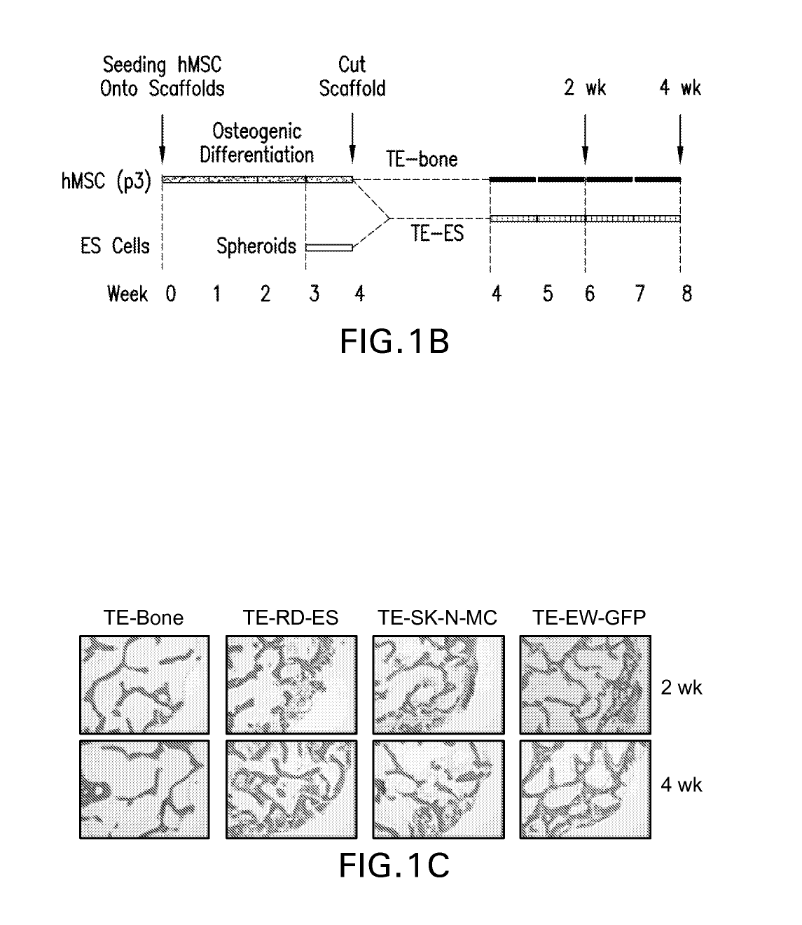

Referring to FIG. 1, tissue engineered models of Ewing's sarcoma according to embodiments of the present disclosure (TE-ES) are illustrated. FIG. 1A depicts a methodology used to develop bioengineered models of Ewing's sarcoma tumor. FIG. 1B depicts TE-ES generation. Fully decellularized bone scaffolds (4 mm diameter×4 mm high plugs) are seeded with hMSCs. After 4 weeks of culture in osteogenic differentiation medium, bone constructs are bisected. One half is seeded with Ewing's sarcoma spheroids (3 per construct); the other half is used as control (TEbone). Both TE-ES and TE-bone are cultured for 2 or 4 weeks in ES medium. FIG. 1C shows Hematoxylin and Eosin images of TE-bone controls and TE-ES models (TE-RD-ES, TE-SK-N-MC, TE-EW-GFP) at week 2 and 4 after introducing tumor spheroids.

Tumor Model To form the tumor model according to some embodiments, Ewing's sarcoma (ES) spheroids (providing a 3D context for local interactions of cancer cells) are introduced into a human bone niche generated by tissue-engineering technology (TE-bone) (FIG. 1A). TE-bone plugs are cultured for 4 weeks in osteogenic differentiation medium. In parallel, tumor spheroids are cultured in ES medium for one week. TEbone plugs are bisected through the center, and 3 ES spheroids are introduced into one half of the construct, generating the Tissue-engineered Ewing's Sarcoma (TE-ES) model; the other half of each TE-bone plug can serve as control. TE-ES models and their control counterparts are cultured for an additional 2 or 4 weeks in ES medium (FIG. 1B). Three different TE-ES models are generated, using various ES cell lines (TE-RD-ES, TE-SK-N-MC, TE-EW-GFP) (FIG. 1C).

Bone Niche hMSCs differentiate into osteoblastic lineage and form viable, functional human bone when cultured on 3D scaffolds made of decellularized bone in osteogenic-differentiation medium. According to an embodiment of the present disclosure, the following approach is used to engineer a bone niche (TE-bone) for the tumor model. First, the osteogenic potential of hMSC is tested after three weeks of monolayer culture in osteogenic medium. Positive Alkaline phosphatase and Von Kossa stainings (FIG. 6A-B) and expression of bone markers by qRT-PCR (FIG. 6C) demonstrates bone differentiation capacity of hMSCs. In parallel, 1.5×10⁶ hMSC (passage 3) are cultured in 4×4mm cylindrical decellularized bone scaffolds for 6 and 8 weeks, in osteogenic differentiation medium, and observed elevated expression levels of bone-related markers (OPN, BSP and OCN) as compared to the differentiation of same cells in monolayer cultures (FIG. 6D). Bone-related protein expression by IHC suggest that TE-bone is properly generated (FIG. 6E). Hypoxia is a pivotal microenvironmental factor for tumor development. Thus, hypoxia is confirmed in the middle of the TE-bone by tissue immunofluorescence of pimonidazole-binding cells (FIG. 6F).

Ewing's sarcoma cells Ewing's sarcoma family of tumors (ESFT) is characterized by aggressive, undifferentiated, round cells, with strong expression of CD99, affecting mostly children and young adults. ESFT comprises of Ewing's sarcoma (ES) that arises in bone, extraosseous ES (EES), peripheral primitive neuroectodermal tumors (pPNET) and Askin's tumors with a neuroectodermal origin. The chromosomal translocation t(11:22)(q24:q212) is the most common mutation (˜85-90% of cases) in ESFT and leads the formation of the EWS/FLI fusion protein which contributes to tumorigenesis in the cells of origin. Analyses of molecular signatures suggest that ESFT originate from mesenchymal and neural crest.

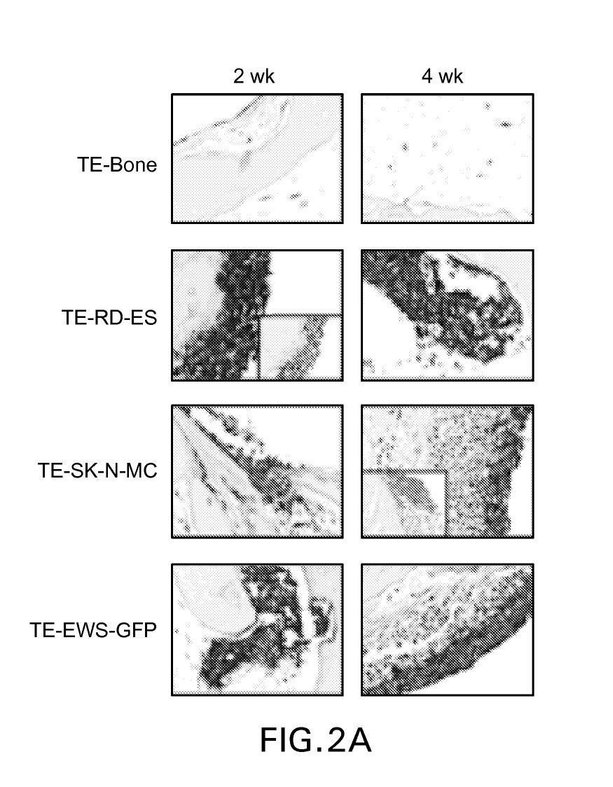

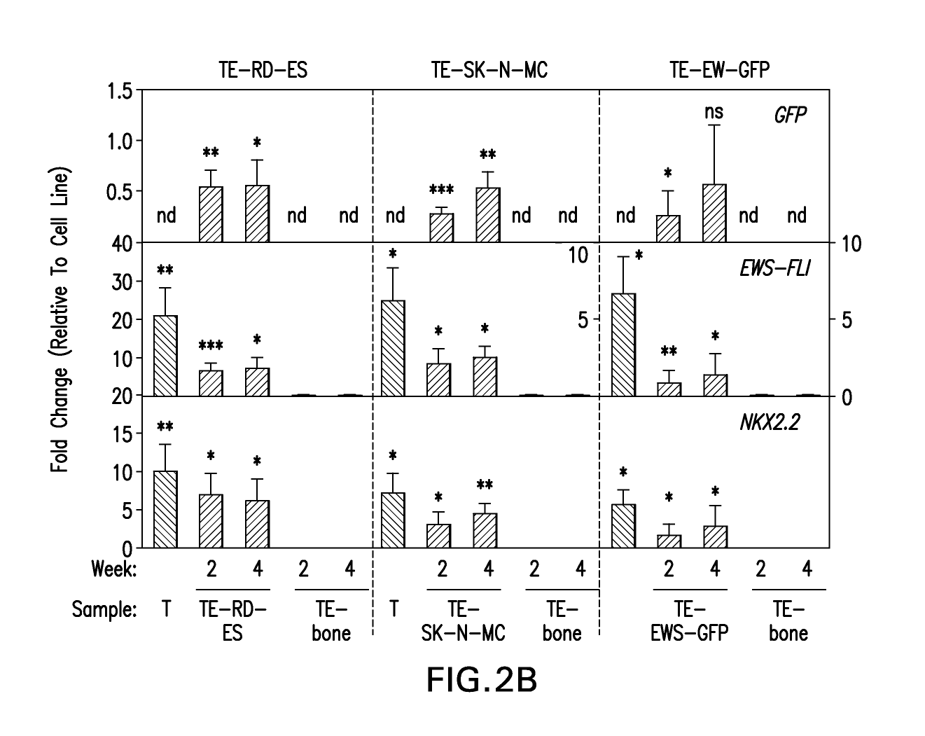

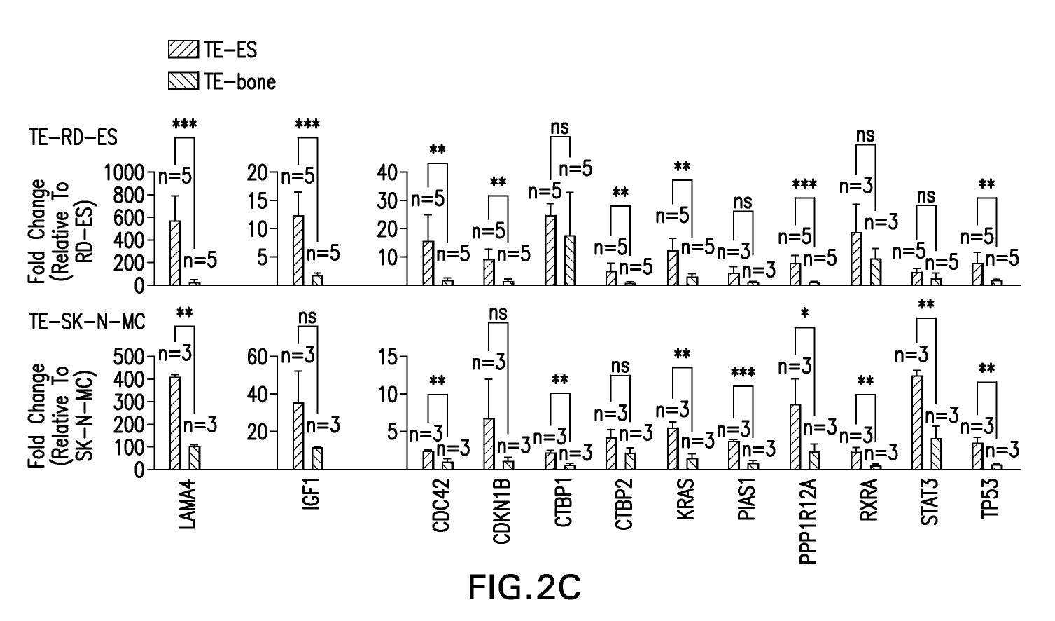

Referring to FIG. 2, characterization of TE-ES models are depicted. In FIG. 2A, Immunohistochemical staining of TE-bone and TE-ES models for Ewing's sarcoma marker CD99 at weeks 2 and 4 are shown. Insets represent negative controls without primary antibody. Representative images are shown (n=3 per condition). Counterstaining is performed with Hematoxylin QS (blue) FIG. 2B depicts qRT-PCR analysis of GFP, EWS-FLI and NKX2.2. FIG. 2C depicts qRT-PCR analysis of the ES genes expressed in tumors and not in cell lines cultured in 2D. In all cases, fold change is calculated by first normalizing to actin levels in the individual samples and then to the corresponding levels in cells cultured in 2D. Data are shown as Average±SD (n=3-5). Two-tailed Student's t-test was used to determine statistical significance. *p<0.05; p<0.01, p<0.001; nd, not determined; ns, not significant; T, Ewing's sarcoma tumors.

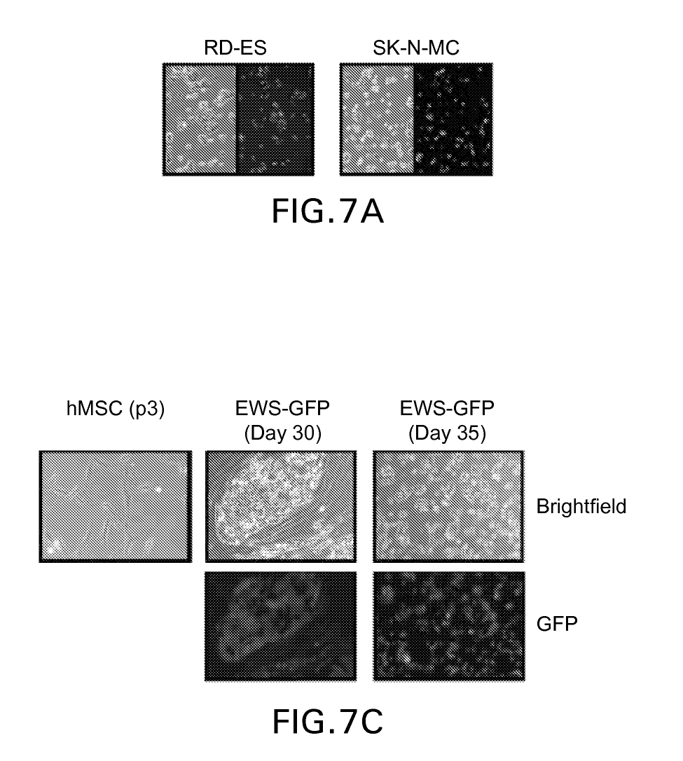

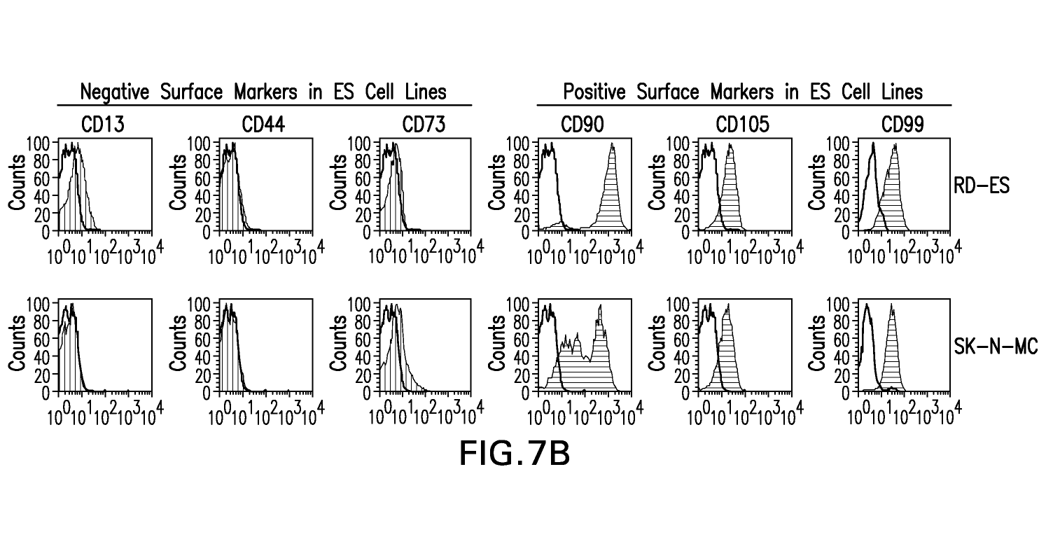

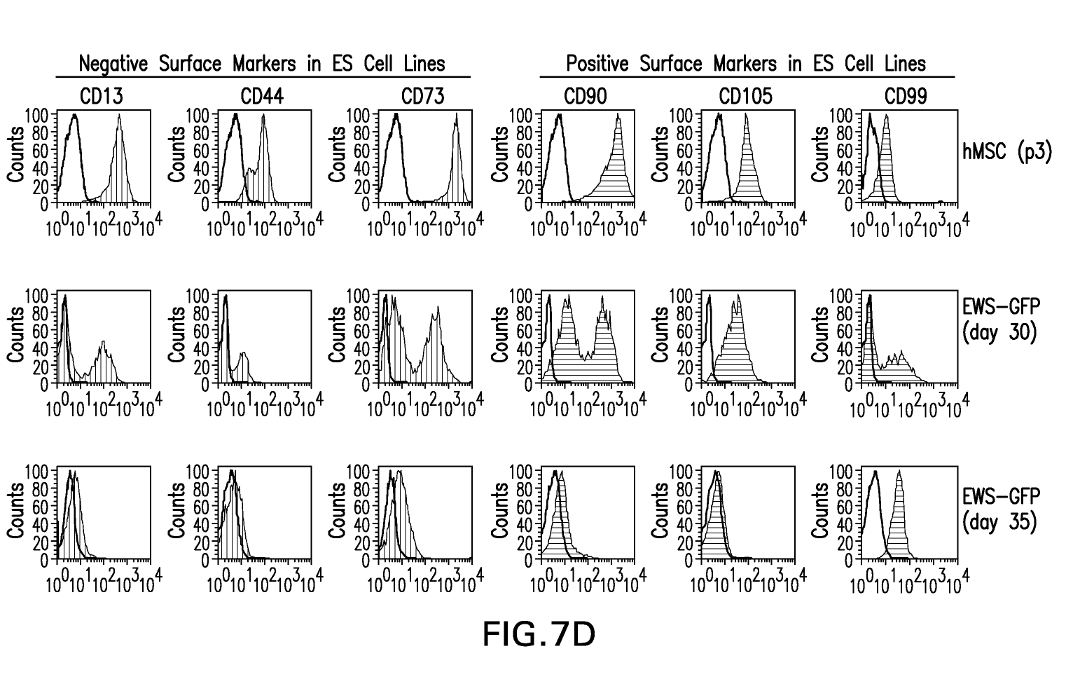

Two Ewing's sarcoma cell lines expressing GFP, RD-ES (primary bone tumor cell line) and SK-N-MC (primary cells originated from an Askin's tumor and metastasizing in the supraorbital area) are used to develop the tumor models (FIG. 7A). Surface markers (characterized by FACS) are CD13, CD44 and CD73 negative and CD90, CD105 and CD99 positive (FIG. 7B). In order to generate in vitro an ES cell line (EW-GFP cell line), a lentiviral plasmid containing the EWS/FLI mutation is introduced into hMSCs (FIG. 7C). Surface proteins expression in EW-GFP cell line (by flow cytometry) is compared to hMSCs, exhibiting high levels of the ES-related marker CD99 and losing CD13, CD44 and CD73 hMSC-specific markers (FIG. 7D).

Re-Expression of Focal Adhesion and Cancer-Related Genes

In order to validate the TE-ES model, histological sections are analyzed by hematoxylin-eosin staining, detecting large areas with small-round cells that were CD99 positive and surrounded by bone cells and ECM (FIG. 2A). GFP levels in TE-ES models and their cell line counterparts cultured in monolayers (by qRT-PCR) confirm expression in both cultures (FIG. 2B), demonstrating ES tissue formation and the presence of ES cells in the bone context. EWS-FLI mRNA and the EWSFLI target NKX2.2 are expressed at low levels in ES cell monolayers as compared to native ES tumors from patients (FIG. 2B). Notably, both genes are up-regulated in all three TE-ES models, for all three cell lines described herein, showing a clear effect of the microenvironment in regulating ES gene profile (FIG. 2B).

Significant differences exist in gene expression between tumors from patients and cells cultured in monolayers, due to the flat, unnatural plastic environment. The presence or absence of expression of genes in 44 tumors from patients and 11 cell lines were analyzed by applying the barcode method to the Affymetrix Human Genome U1332 Plus 2 gene expression data of Savola et al.

599 genes are identified that were expressed in tumors but not in cell lines (Table 1). Comparing mRNA expression between the two cell lines (RD-ES and SK-N-MC) and 3 ES tumors by qRT-PCR, upregulation of 24 genes in ES tumors is confirmed. All these genes are related to focal adhesion and pathways in cancer (Table 2; FIGS. 8, 9, 10 and 11). Analysis of these 24 genes in the TE-RD-ES and TE-SK-N-MC models relatively to their monolayer counterparts, confirms strong re-expression (fold change >3) for 12 genes (FIG. 2C).

IGF1 is one of the targets found and validated (12.2±4.11 fold change in TE-RD-ES relative to RD-ES cell monolayers; 35.08±16.84 fold change in TE-SKN-MC relative to SK-N-MC monolayers). IGF signal transduction pathway is thought to play a key role in ESFT development and proliferation. These results support the importance of tumor microenvironment for gene expression and suggest that TE-ES models recapitulate, at least in part, ES gene expression signatures.

Recapitulation of the Hypoxic and Glycolytic Tumor phenotype

At early stages of cancer, tumors are avascular masses where oxygen and nutrients delivery are supplied by diffusion and therefore, growing in central areas is compromised. To maintain energy production, tumor cells respond and adapt to the hypoxic environment by increasing the amount of glycolytic enzymes and glucose transporters, such as GLUT1 and GLUT3, via the hypoxia-inducible factor-1 (HIF1α). Studies using tumor spheroids and tumor micro-regions in vivo, show an outer viable tumor (with proliferating cells), an inner hypoxic area (with quiescent adapted viable cells) and a central necrotic core where oxygen and glucose levels are critically low. The tumor model provides a native-like niche that mimics tumor heterogeneity in terms of oxygen and nutrients supply, as demonstrated by hypoxia in the center of the tissue constructs, but not in the outer areas (FIG. 6F).

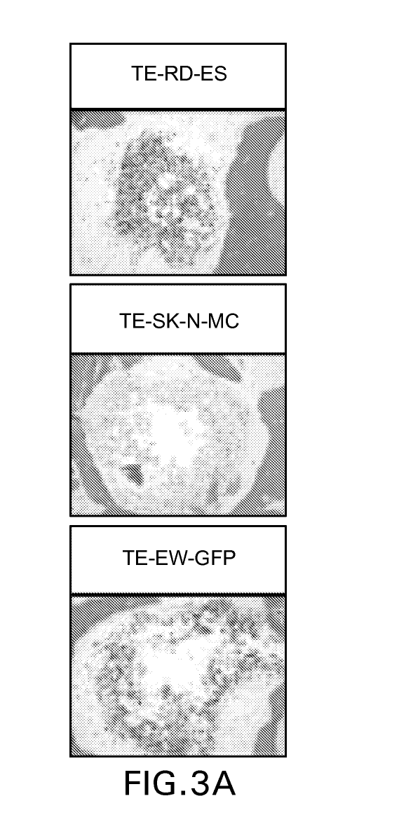

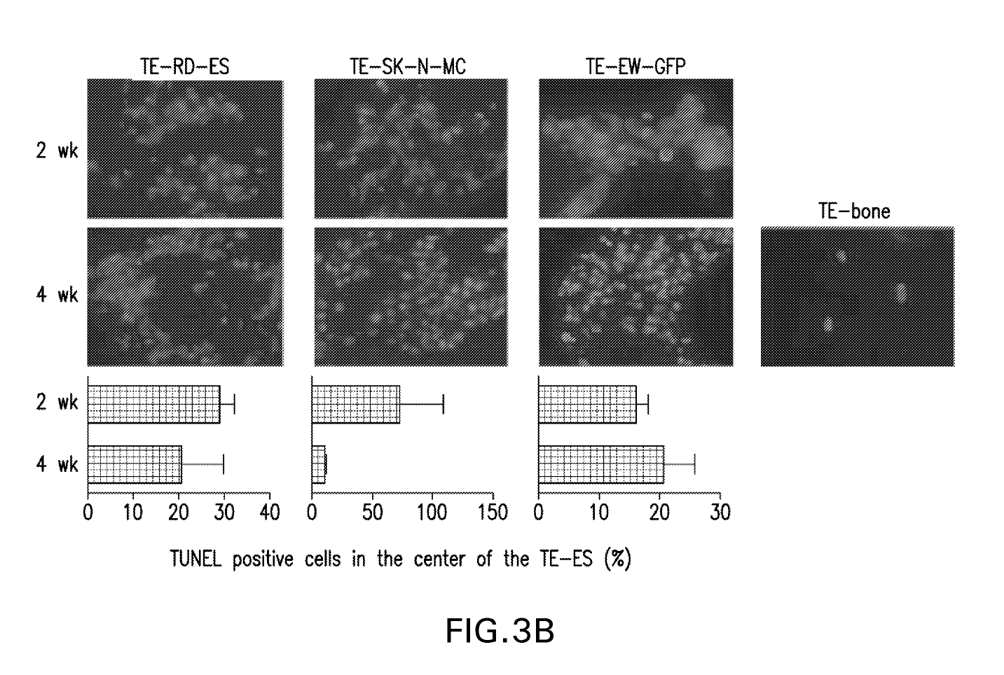

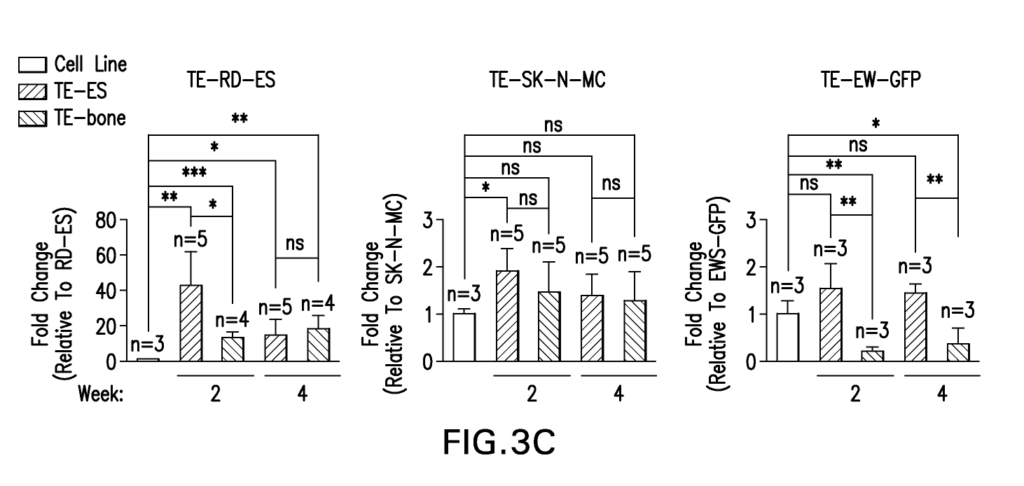

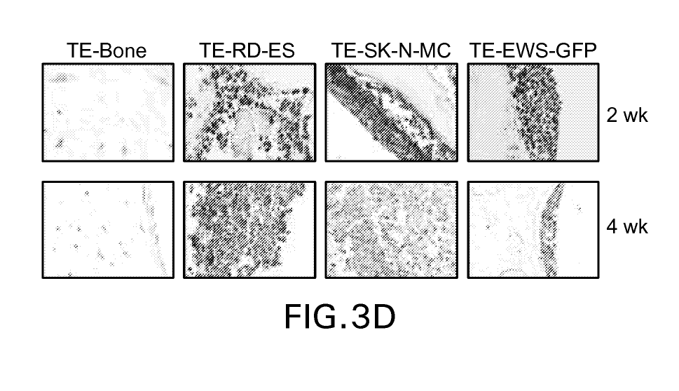

Referring to FIG. 3, expression of hypoxic and glycolytic tumor phenotypes are depicted. FIG. 3A shows Necrotic areas in the inner part of TE-ES models identified by Hematoxylin and Eosin staining of TE-RDES, TE-SK-N-MC and TE-EW-GFP at week 2. Representative images are shown (n=3 per condition). FIG. 3B shows HIF1α mRNA levels in TE-ES models. Fold change is calculated by first normalizing to actin levels in the individual samples and then to the corresponding levels in cells cultured in 2D. Data are shown as Average±SD (n=3-5). Statistical significance is determined by the two-tailed Student's t test. *p<0.05; p<0.01, p<0.001; ns, not significant. FIG. 3C TUNEL immunofluorescent staining of TE-ES and TE-bone in the center on the models. Upper panel: representative pictures of TUNEL-stained inner areas. Apoptotic cells stain red; cell nuclei were stained by Hoechst 33342. Lower panel: Quantification of TUNELpositive cells in the inner part of the indicated TE-ES models. FIG. 3D shows Immunohistochemical staining of GLUT-1 in the indicated TE models over time. Counterstain: Hematoxylin QS (blue). Representative images are shown (n=3 per condition).

In order to evaluate whether TE-ES models recapitulate the initial steps of tumor generation, necrotic areas in the core of the tumor models were analyzed and compared the levels of HIF1α and GLUT1 to those in cell monolayers and TE-bone controls. First, focus on the construct interiors revealed necrotic areas similar to those observed in native tumors (FIG. 3A). TUNEL assays after 4 weeks of cultivation revealed higher cell death in the middle of the TE-SK-N-MC tumor model (73±36%) relatively to TE-RD-ES (29±3%) and/or TE-EW-GFP(16±2%) (FIG. 3B). These results suggest that RD-ES and EW-GFP cell lines may be better adapted than SK-N-MC cell line to restrictive conditions at the centers of the constructs.

In response to hypoxia (at week 2), transcription levels of HIF1α were 40 times higher in the TE-RD-ES tumor model relatively to the RD-ES cell monolayers, and 30 times higher relatively to TE-bone. HIF1α expression decreased with time in culture, reaching at week 4 levels similar to those in TE-bone (FIG. 3C). Transcriptional expression of HIF1α was not significantly increased by hypoxia in TE-SK-N-MC and TE-EW-GFP models as compared to cell lines (FIG. 3C). Also, the SK-N-MC and EW-GFP cell lines express higher levels of HIF1α than the RD-ES line, and the expression levels in the SK-N-MC cells were comparable to those in TE-bone. These data suggest that tumor cells that have low transcriptional levels of HIF1α (RD-ES line) increase expression in order to adapt to hypoxic environment. In contrast, cell lines expressing high levels of HIF1 (SK-N-MC and EW-GFP) seem to be insensitive to hypoxia, at least at the transcriptional levels. HIF1α thus appear to play a protective role in the adaptation of tumor cells to hypoxia.

To assess the role of hypoxia in the induction of glycolytic response, the levels of GLUT1 protein in TE-bone and TE-ES models were examined. Very high levels of GLUT1 are observed favoring glucose uptake and tumor survival in inner areas where oxygen and medium supply are compromised (FIG. 3D). GLUT1 was expressed in necrotic areas in the TE-SK-N-MC model.

Taken together, these data demonstrate that the RD-ES cells expressing high levels of HIF1α adapt to hypoxia in the TE bone environment by recapitulating some aspects of hypoxic and glycolytic tumor phenotype, and mimicking inner-necrotic and outersurvival signatures. In comparison, the SK-N-MC and EW-GFP cells expressing low levels of HIF1α show less ability to adapt to hypoxic microenvironment.

Recapitulation of Angiogenic Ability and Vasculogenic Mimicry.

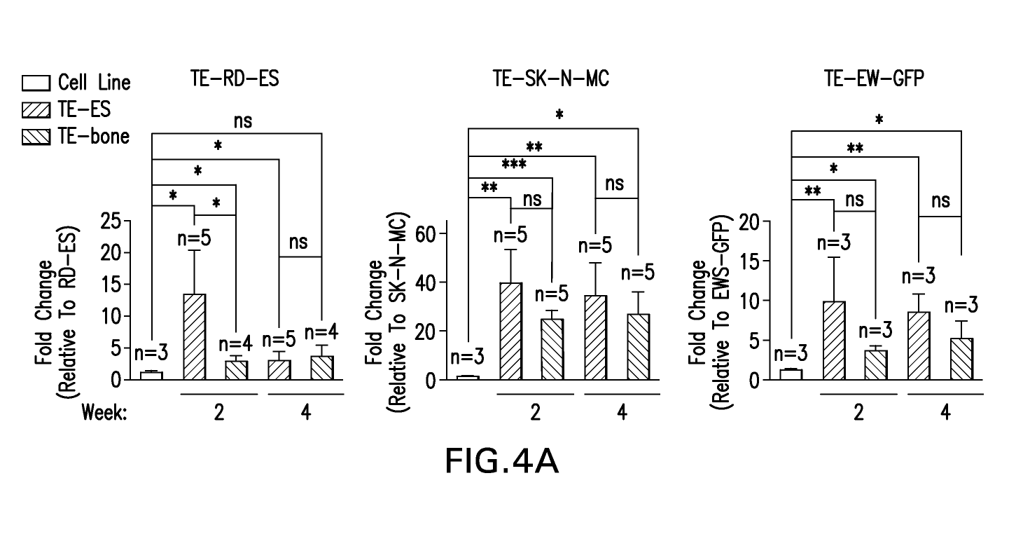

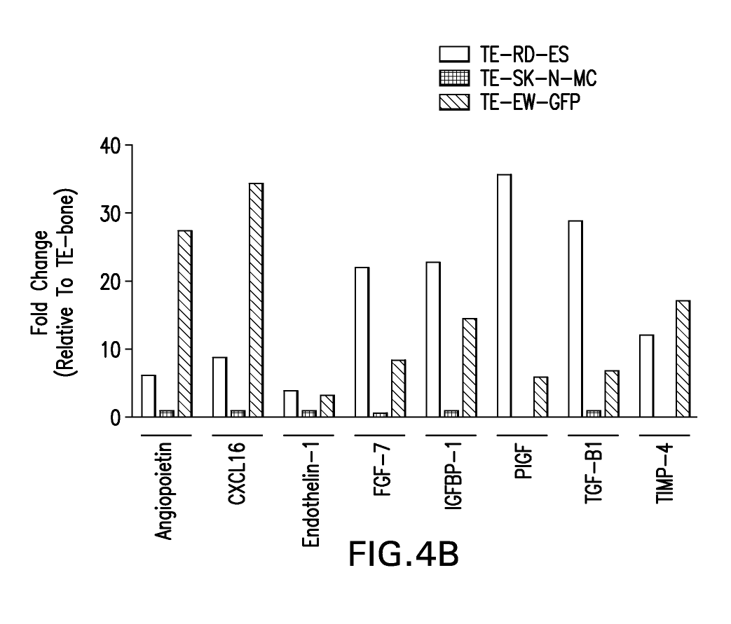

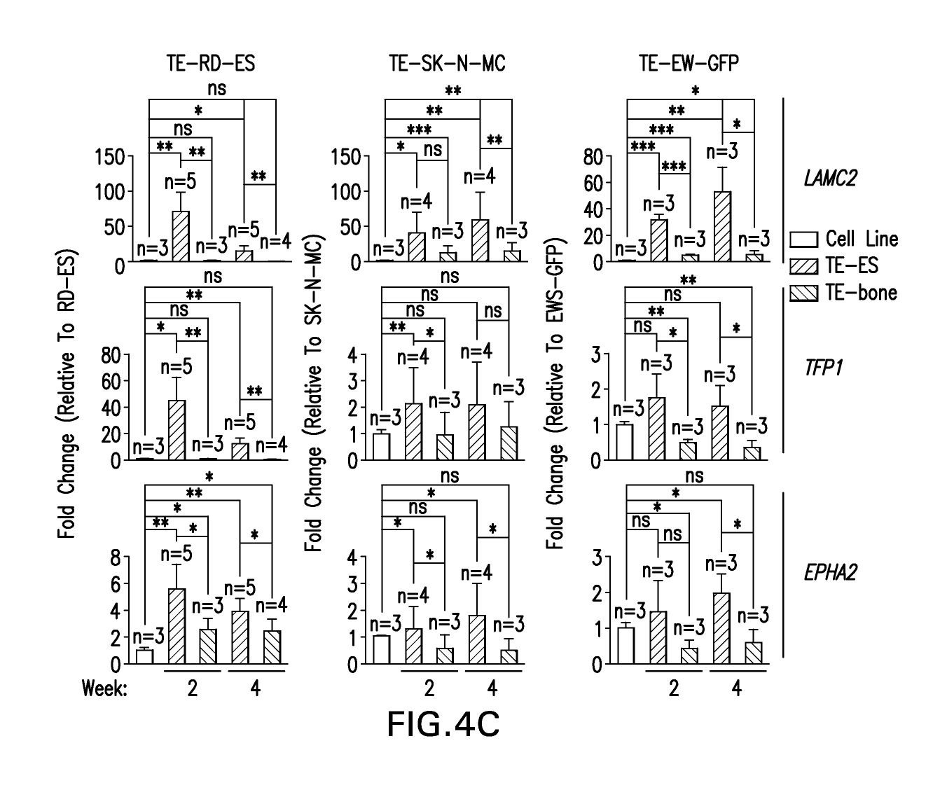

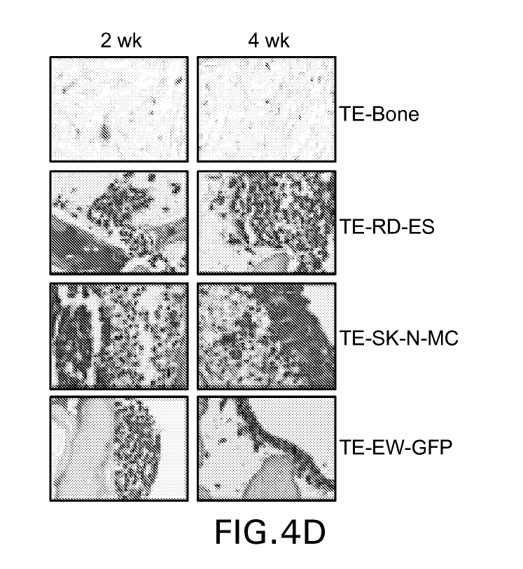

Referring to FIG. 4, angiogenesis and vasculogenic mimicry are depicted. FIG. 4A shows VEGFa mRNA levels in TEES models. Fold change is calculated by first normalizing to actin levels in the individual samples and then to the corresponding levels in cells cultured in 2D. Data are shown as Average±SD (n=3-5). Two-tailed Student's t-test is used to determine statistical significance. *p<0.05; p<0.01, p<0.001; ns, not significant. FIG. 4B shows Angiogenesis-related proteins detection in TE-ES culture media. Expression levels of the indicated proteins were assessed by ELISA and compared with expression levels in the TE-bone counterparts. FIG. 4C shows qRT-PCR analysis of vasculogenic mimicry markers. Relative endogenous expression of each gene was normalized to actin and the fold change was obtained normalizing to the levels in corresponding cell lines cultured in 2D. Data are shown as Average±SD (n=3-5). Statistical significance was determined by the two-tailed Student's t test. *p<0.05; p<0.01, p<0.001; ns, not significant. FIG. 4D shows representative images of PAS-stained sections from TE-bone and TE-ES models at week 2 and 4. Representative images are shown (n=3 per condition).

Tumor cells respond to oxygen and nutrient deprivation by promoting vascularization that maintains tumor growth and survival. Induction of vascular endothelial growth factor (VEGF-a) is an essential feature of tumor angiogenesis that is driven by hypoxia and mediated by HIF1α. To address whether hypoxia modulates angiogenic ability of the tumor, VEGF-a transcriptional levels in TE-ES models were analyze. High induction of VEGF-a in TE-RD-ES were found at week 2 compared to the RD-ES cell line and TE bone (FIG. 4A). Notably, levels decreased by week 4, as observed for HIF1-α. In further support of the adaptive advantage of RD-ES cells cultured in TE-bone, VEGF-a mRNA levels were not significant increased in TE-SK-N-MC and TE-EW-GFP tumor models as compared to TE-bone controls (FIG. 4A).

Then, angiogenic proteins secreted by TE-ES tumors are identified. By ELISA analysis of 24-hr supernatants, 56 human angiogenesis-related proteins were analyzed at week 2. Due to the differences in growth of different cell lines, it was not possible to directly compare secretion rates. However, these analyses clearly demonstrated that 8 proteins (Angiopoietin, CXCL16, Endothelin-1, FGF-7, IGFBP1-1, PIGF, TGF-B1 and TIMP4) were highly expressed in TE-RD-ES and TE-EW-GFP tumor models compared to TE-bone (fold change >3) In contrast, none of these proteins was detected in the TE-SK-N-MC tumor model. These results confirm that the SK-N-MC cells failed to induce essential adaptive elements to survive and proliferate in TE-bone (FIG. 4B). Interestingly, Endothelin-1 is implicated in ES proliferation and invasion while IGFBP1-1 prolongs the half-life of IGF-1, a well-known target gene of EWS-FLI and TGF-β1. These observations are consistent with previous studies, validating the current system.

Finally, vasculogenic mimicry (VM) is evaluated in TE-ES models. Native ES is featured by the presence of blood lakes and PAS positive cells expressing endothelium-associated genes. This property is known as VM and is stimulated by hypoxia. Thus, VM can provide functional perfusion channels composed only of tumor cells. The endothelium-associated genes (LAMC2, TFPI1 and EPHA2) were highly expressed in the TE-RD-ES at weeks 2 and 4 (FIG. 4C), confirming VM in the TE-RDES model.

Consistent with all other data, cells in the SK-N-MC model re-expressed VM genes as levels lower than those measured for the TE-RD-ES model. However, these expression levels were significantly upregulated at week 2 for TFP1 (p<0.01) and EPHA2 (p<0.05) and at week 4 for LAMC2 (p<0.01) and EPHA2 (p<0.05) as compared to SK-N-MC and TE-bone (FIG. 4C). Moreover, the TE-EW-GFP model expressed high levels of LAMC2, TFPI1 and EPHA2 at week 2 and 4 as compared to TE-bone (FIG. 4C). Tissue sections stained with PAS revealed positive areas in all the TE-ES models (except in TE-EW-GFP at week 2), as compared to negative-PAS TEbone (FIG. 4D). Taken together, these results confirm that RD-ES cell line has higher capability to adapt to TE-bone than the SK-N-MC line.

According to various embodiments of the present disclosure, human tumor models predictive of native tumors in vitro are provided. Spheroids of tumor cells and porous scaffolds capture 3D aspects with control of oxygen, tension, and pH. Cancer is a complex disease where interactions between tumor cells and non-neoplastic cells play an important role in carcinogenesis. Herein, various embodiments provide models of human tumors, by incorporating Ewing's sarcoma cell spheroids into a bioengineered tridimensional bone niche, and thus enabling multiple interactions of tumor cells with other tumor cells, bone tissue matrix and bone cells.

Tumor cell lines cultured in 2D lose their transcriptional profiles and downregulate many genes implicated in cell-cell and cell-ECM interactions, such as focal adhesion genes. Gene expression profiles of cell lines cultured in monolayers are compared with native tumors, with focus on differentially expressed focal adhesion genes and cancer pathways. The induction of 12 genes in both TE-RD-ES and TE-SK-N-MC models evidence a major role of microenvironment in the acquirement of tumor expression profile. Models according to the present disclosure can thus be used for characterization of differentially expressed genes and help identify new tumor targets. As discussed above, induction of CDC42 and PPP1R12A is observed, both of which are related to Rho family of GTPases. Inhibition of some Rho pathway members through therapeutic compounds is applied in preclinical studies suggesting that CDC42 and PPP1R12A are potential candidates for ES therapy.

The bone niche has an important role in acquiring ESFT features to tumor cells, such as hypoxic and glycolytic phenotypes, angiogenesis potential and vasculogenic mimicry. The three ES cell lines discussed herein exhibit different behaviors in the bioengineered tumor model of the present disclosure. The primary bone tumor RD-ES cell line mimics ESFT signature, the in vitro-generated EWS-GFP cell line only in part and the metastatic SK-N-MC cell line was not able to recapitulate many of the tumor characteristics. These differences correlate to the expression levels of HIF1α (low in RD-ES cells, and high in SK-N-MC and EW-GFP cells), suggesting that HIF1α plays a protective role in the adaptation of tumor cells to hypoxia.

According to various embodiments of the present disclosure, tumor cells are studied within the 3D niche engineered to mimic the native host tissue. In various embodiments, the inclusion of stromal cells is provided, and tumor microvasculature and fine-tuned control of oxygen and nutrients are provided through the use of perfusion bioreactors.

### EXAMPLES

Native Tumors

Ewing's sarcoma tumors were obtained from a Tissue Bank. The samples were fully de-identified. Three different frozen tissue samples were cut in sets of 6 contiguous 10 μm-thick sections and homogenized in Trizol (Life technologies) for RNA extraction and subsequent gene expression analysis.

Cell Culture

Ewing's sarcoma cell lines SK-N-MC (HTB-10) and RD-ES (HTB-166) were purchased and cultured according to the manufacturer's specifications. RD-ES cells were cultured in ATCC-formulated RPMI-1640 Medium (RPMI) and SK-N-MC cells were cultured in ATCC-formulated Eagle's Minimum Essential Medium (EMEM). Both media were supplemented with 10% (v/v) Hyclone FBS and 1% penicillin/streptomycin. EWS-GFP cells were cultured in DMEM supplemented with 10% (v/v) Hyclone FBS and 1% penicillin/streptomycin).

U2OS osteosarcoma cell line and HEK293T cell line were provided and cultured in Dulbecco's Modified Eagle Medium (DMEM) supplemented with 10% (v/v) Hyclone FBS and 1% penicillin/streptomycin).

The cultivation, seeding and osteogenic differentiation of Human Mesenchymal Stem Cells (hMSC) were performed. Briefly, hMSC were cultured in basic medium (DMEM supplemented with 10% (v/v) Hyclone FBS and 1% penicillin/streptomycin) for maintenance and expansion, followed by osteogenic medium (basic medium supplemented with 1 μM dexamethasone, 10 mM β-glycerophosphate, 50 μM ascorbic acid-2-phosphate) for osteogenic differentiation. Due to the highly osteogenic properties of the mineralized bone scaffolds used to culture the cells, the supplementation of MBP-2 was not necessary.

All cells were cultured at 37° C. in a humidified incubator at 5% CO₂.

Retroviral and Lentiviral Transductions

Retroviral transductions were performed using a GFP retroviral vector (pBabe-Puro-GFP). Lentiviral transductions were performed. EWS-FLI-GFP expression vector was provided.

Tumor Cell Spheroids

To form tumor cell spheroids, 0.3×10⁶ Ewing's sarcoma cells were centrifuged in 15 mL Falcon tubes, 5 minutes at 1200 rpm, with 4 mL of medium and cultured for one week at 37° C. in a humidified incubator at 5% CO₂.

Tissue engineered model of tumor Cell culture scaffolds (4 mm diameter×4 mm high plugs) were prepared from fully decellularized bone. The scaffolds were seeded with 1.5×10⁶ hMSCs (passage 3) and cultured in 6 mL of osteogenic medium for 4 weeks. Medium was changed biweekly. After 4 weeks, the scaffolds were bisected; one half was seeded with Ewing's sarcoma cells (3 spheroids per scaffold) (TE-ES) and the other half was used as a control (TE-bone).

Three tumor models were formed using the three tumor cell lines. For each tumor, TE bone was used as a control. TE-RD model (and their counterpart TE-bone controls) were cultured in RPMI medium. TE-SK-N-MC model (and their counterpart TE-bone controls) were cultured in EMEM. TE-EWS-GFP model (and their counterpart TE-bone controls) were cultured in DMEM.

All culture media were supplemented with 10% (v/v) Hyclone FBS and 1% penicillin/streptomycin. TE-ES and TE-bone models were cultured at 37° C. in a humidified incubator at 5% CO₂ for 2 and 4 weeks.

Cytometry

Surface markers analysis by FACS was carried out. hMSC and ES cell lines (RD-ES, SK-N-MC and EWS-GFP) were harvested, centrifugated and incubated at 4° C. for 1 h with fluorochrome conjugated antibodies APC Mouse anti-human CD13 (BD Pharmingen, 557454), APC Mouse anti-human CD44 (BD Pharmingen, 560532), APC Mouse anti-human CD73 (BD Pharmingen, 560847), APC Mouse anti-human CD90 (BD Pharmingen, 559869) and APC Mouse anti-human CD105 (BD Pharmingen, 562408). Negative control cells were stained with APC mouse IgG1, k isotype control, Clone MOPC-21 (BD Pharmingen, 555751). CD99 expression was assed incubating cells with CD99 primary antibody (Signet antibodies, SIG-3620). FACS data were analyzed using FlowJo software version 7.6 (Tree Star Inc., Ashland, Oreg., USA)

Quantitative Real-Time PCR (qRT-PCR).

Total RNA was obtained using Trizol (Life Technologies) following the manufacturer's instructions. RNA preparations (2 μg) were treated with “Ready-to-go you-prime first strand beads” (GE Healthcare) to generate cDNA. Quantitative real-time PCR was performed using DNA Master SYBR Green I mix (Applied Biosystems). mRNA expression levels were quantified applying the ΔCt method, ΔCt=(Ct of gene of interest—Ct of Actin).

GFP primers were selected. Other qRT-PCR primer sequences were obtained from the PrimerBank data base (http://pga.mgh.harvard.edu/primerbank/):

| Gene Description | PrimerBank ID |
|---|---|
| beta actin (Actin) | 4501885a1 |
| EWS-FLI1 fusion isoform type 8 (EWS-FLI) | 633772a1 |
| Homo sapiens NK2 homeobox 2 (NKX2-2) | 32307133b1 |
| Homo sapiens tumor protein p53 (TP53) | 371502118c1 |
| ACTN4 Homo sapiens actinin, a 4 (ACTN4) | 316660986c2 |
| CCND2 Homo sapiens cyclin D2 (CCND2) | 209969683c1 |
| COL1A2 Homo sapiens collagen, type I, α2 (COL1A2) | 48762933c3 |
| COL3A1 Homo sapiens collagen, type III, α1 | 110224482c2 |
| (COL3A1) | |
| Homo sapiens collagen, type VI, a1 (COL6A1) | 87196338c2 |
| COL6A2 Homo sapiens collagen, type VI, α2 | 115527065c1 |
| (COL6A2) | |
| COL6A3 Homo sapiens collagen, type VI, α3 | 240255534c1 |
| (COL6A3) | |
| FLNB Homo sapiens filamin B, β (FLNB) | 256222414c2 |
| MYLK Homo sapiens myosin light chain kinase (MYLK | 116008189c1 |
| Homo sapiens 3-phosphoinositide dependent protein | 60498971c1 |
| kinase-1 (PDPK1) | |
| Homo sapiens protein phosphatase 1, regulatory | 219842213c1 |
| subunit 12A (PPP1R12A) | |
| Homo sapiens insulin-like growth factor 1 | 163659898c1 |
| (somatomedin C) (IGF1) | |
| VCL Homo sapiens vinculin (VCL) | 50593538c1 |
| CDKN1B Homo sapiens cyclin-dependent kinase | 207113192c3 |
| inhibitor 1B (p27, Kip1) (CDKN1B) | |
| Homo sapiens C-terminal binding protein 1 (CTBP1) | 61743966c2 |
| CTBP2 Homo sapiens C-terminal binding protein 2 | 145580576c1 |
| (CTBP2) | |
| ETS1 Homo sapiens v-ets erythroblastosis virus | 219689117c1 |
| E26 oncogene homolog 1 (avian) (ETS1) | |
| c-K-ras2 protein isoform a (KRAS) | 15718763a1 |
| PIAS1 Homo sapiens protein inhibitor of activated | 7706636c2 |
| STAT, 1 (PIAS1) | |
| Homo sapiens retinoid X receptor, alpha (RXRA) | 207028087c3 |
| Homo sapiens signal transducer and activator of | 47080104c1 |
| transcription 3 (STAT3) | |
| Homo sapiens cell division cycle 42 (GTP binding | 89903014c1 |
| protein, 25 kDa) (CDC42) | |
| Homo sapiens collagen, type IV, α2 (COL4A2) | 116256353c1 |
| Homo sapiens catenin (cadherin-associated | 148233337c2 |
| protein), β1, 88 kDa (CTNNB1) | |
| Homo sapiens jun proto-oncogene (JUN) | 44890066c1 |
| laminin a 4 chain (LAMA4) | 4504949a2 |
| Homo sapiens laminin, β 1 (LAMB1) | 167614503c1 |
| Homo sapiens laminin, γ1 (formerly LAMB2) (LAMC1) | 145309325c3 |
| Homo sapiens phosphoinositide-3-kinase, regulatory | 335057530c3 |
| subunit 1 (a) (PIK3R1) | |
| Homo sapiens phosphatase and tensin homolog (PTEN) | 110224474c2 |
| Homo sapiens hypoxia inducible factor 1, α subunit | 194473734c1 |
| (HIF1A) | |
| Homo sapiens vascular endothelial growth factor A | NM_001101 |
| (VEGFA) | |
| Homo sapiens EPH receptor A2 (EPHA2) | 296010835c1 |
| Homo sapiens tissue factor pathway inhibitor (TFPI) | 98991770c1 |
| Homo sapiens laminin, γ2 (LAMC2) | 157419139c1 |

Microarray data analysis. Expression of genes in native Ewing's Sarcoma tumors and cell lines was studied in 11 cell lines and 44 tumors by applying the barcode method to the Affymetrix Human Genome U1332 Plus 2 gene expression data. A probeset was considered expressed in cell lines/tumors only if detected in all cell lines/tumors. Where a gene had multiple probesets, the gene was only counted once. Genes expressed in cell lines, but not tumors, or in tumors, but not cell lines, were identified from the asymmetric difference of both sets.

Histology and Immunohistochemistry (IHC).

TE-ES and TE-bone models were fixed in 10% formalin, embedded in paraffin, sectioned at 4 μm and stained with hematoxylin and eosin (H/E). The sections were then stained for CD99 (dilution 1:500; Signet antibodies, SIG-3620) and GLUT1 (dilution 1:500; Abcam, ab652) as previously described, and counterstained with Hematoxylin QS (Vector Labs). For PAS staining, periodic acid-Schiff (PAS) (from Sigma-Aldrich) was used according to the manufacturer's instructions.

hMSC (passage 3) were plated in 24 well plates (1×10⁴ cells/cm²) and cultured for 3 weeks in either basic medium or osteogenic medium. At weeks 1, 2 and 3 osteogenic differentiation was analyzed by alkaline phosphatase activity (Sigma-Aldrich, St Louis, Mo., USA), following the manufacturer's instructions and by von Kossa staining Sections were incubated with 1% AgNO3 solution in water and exposed to a 60 W light for 1 h.

Hypoxyprobe™-1 (pimonidazole) Kit for the Detection of Tissue Hypoxia (Chemicon International, Inc., Temecula, Calif., USA) was used to detect hypoxia in TE-bone according to the manufacturer's instructions. Preparations were mounted with vectashield and Nuclei were counterstained with DAPI (Vector Labs, H-1200).

TUNEL assay. Apoptotic cells were detected by an in situ cell death detection kit, TMR red (Roche Applied Science, Mannheim, Germany), according to the manufacturer's instructions. The assay measures DNA fragmentation by immunofluorescence using TUNEL (terminal deoxynucleotidyl transferase-mediated dUTP nick end-labeling) method at the single cell level. One hundred cells per field (n=3) in the center of the TEES model (n=3) were counted to quantify the percentage of apoptotic cells. Nuclei were stained with Hoechst 33342 (Molecular probes).

Enzyme-Linked Immunoabsorbent Assay (ELISA)

24-hour supernatants from TE-ES and TE-bone controls were analyzed to detect angiogenic proteins, using a Proteome Profiler Human Angiogenesis Array Kit (R&D Systems, ARY007) according to the manufacturer's instructions.

With regard to FIG. 6, generation and characterization of TE-bone is illustrated. In FIG. 6A Osteogenic differentiation evidenced by Alkaline phosphatase staining hMSCs in monolayer were cultured in hMSC medium or osteogenic medium for 3 weeks. Alkaline phosphatase staining was performed at week 1, 2 and 3 as described in supplementary methods. Differentiated stem cells positive for alkaline phosphatase were stained blue. Images are representative of n=3 samples per condition. FIG. 6B shows Mineral deposition analysis by the von Kossa method. hMSC were cultured as specified in FIG. 6A. Black stained phosphate deposits demonstrated osteogenic differentiation of hMSC. Images are representative of n=3 samples per condition. FIG. 6C shows qRT-PCR analysis of bone genes during osteogenic differentiation in monolayer. mRNA levels of Osteopontin (OPN), Bone Sialoprotein (BSP), and Osteocalcin (OCN) in hMSC cultured in monolayer in hMSC medium or osteogenic differentiation medium were assessed to demonstrate osteogenic induction and bone differentiation. Data are shown as Average+SD (n=3) FIG. 6D shows qRT-PCR analysis of bone genes during osteogenic differentiation in scaffold. mRNA levels of Osteopontin (OPN), Bone Sialoprotein (BSP), and Osteocalcin (OCN) in hMSC cultured in a bone scaffold for 6 and 8 weeks in osteogenic differentiation medium were assessed and compared to hMSC at t=0.

FIG. 6E shows Bone-related protein expression analysis by IHC in TE-bone at week 8. Counterstaining was performed with hematoxylin QS (blue). Representative images are shown (n=3); H/E, Hematoxylin and Eosin. FIG. 6F shows Hypoxia analysis of TE-bone by tissue immunofluorescence of pimonidazole-binding cells (green). Nuclei were stained with DAPI. Representative images are shown (n=3 per condition).

Referring to FIG. 7, charactarization of Ewing's sarcoma cell lines is illustrated. FIG. 7A shows Morphology of the ES cell lines RD-ES and SK-N-MC. Left panel: brightfield images showing typical small round cell morphology. Right panel: GFP expression images by fluorescence microscopy. RD-ES and SK-N-MC were stably transduced with pBabe-GFP retroviral vector as described in supplementary methods. FIG. 7B shows FACS analysis of negative and positive surface markers in Ewing's sarcoma cells. FIG. 7C shows Top panels: brightfield images of hMSC (passage 3) and transduced with EWS-GFP vector at day 30 (without passage) and day 35 (passage 2). Low panels: GFP expression images at day 30 and 35 post-transduction. FIG. 7D shows Analysis of hMSC and ES surface markers in EW-GFP cell line. hMSC were CD13, CD44, CD90 and CD105 positive and expressed low levels of the ES-specific CD99 marker. EWS-GFP at day 35 lost hMSC surface proteins, acquiring ES surface markers and expressing high levels of CD99.

Tables 1 and 2 illustrate genes differentially expressed in Ewing's sarcoma tumors and cell lines. Table 1: Number of genes expressed in ESFT and in cell lines. Table 2: Focal adhesion genes and related to pathways in cancer genes expressed in ESFT but not in cell lines.

TABLE 1

| Condition | Number of genes |
|---|---|
| Genes expressed in cell-lines | 2977 |
| Genes expressed in tumors | 2430 |
| Genes expressed in cell-lines but not tumors | 1312 |
| Genes expressed in tumors not cell-lines | 599 |

TABLE 2

| | |
|---|---|
| Focal adhesion: | ACTN4, CCND2, COL1A2, COL3A1, COL6A1, |
| | COL6A2, COL6A3, FLNB, MYLK, PDPK1, |
| | PPP1R12A, IGF1, VCL |
| Pathways in | CDKN1B, CTBP1, CTBP2, ETS1, KRAS, PIAS1, |
| cancer: | RXRA, STAT3, TP53 |
| Both: | CDC42, COL4A1, COL4A2, CTNNB1, FN1, JUN, |
| | LAMA4, LAMB1, LAMBC1, PIK3R1, PTEN |

Referring to FIG. 8, focal adhesion genes and cancer genes expressed in Ewing's sarcoma tumors and bone but not in cell lines are illustrated. qRT-PCR data are shown for two Ewing's sarcoma cell lines (RD-ES and SK-N-MC), three Ewing sarcoma tumors (ESFT) and one osteosarcoma cell line unrelated to ESFT, as control of bone tumor cell line. Relative endogenous expression of each gene was normalized to actin (Average±SD, n=3).

Referring to FIG. 9, focal adhesion genes differentially expressed in Ewing sarcoma tumors and cell lines are illustrated. qRT-PCR analysis of focal adhesion genes expressed in Ewing sarcoma tumorsESFT but not in cell lines. Data are shown for two Ewing's sarcoma cell lines (RD-ES and SK-N-MC), three Ewing sarcoma tumors (ESFT) and one osteosarcoma cell line as control of bone tumor cell line but unrelated to ESFT. Relative endogenous expression of each gene was normalized to actin (Average±SD, n=3).

Referring to FIG. 10, cancer related genes differentially expressed in Ewing sarcoma tumors and cell lines are illustrated. qRT-PCR analysis of cancer related genes expressed in Ewing sarcoma tumors (ESFT) but not in cell lines. Data are shown for two Ewing's sarcoma cell lines (RD-ES and SK-N-MC), three tumors (ESFT) and one osteosarcoma cell line unrelated to ESFT as control of bone tumor cell line. Relative endogenous expression of each gene was normalized to actin (Average±SD, n=3).

Referring to FIG. 11, focal adhesion and Cancer related genes differentially expressed in Ewing sarcoma tumors and cell lines are illustrated. qRT-PCR analysis of cancer related genes expressed in ESFT but not in cell lines. Data are shown for two Ewing's sarcoma cell lines (RD-ES and SK-N-MC), three tumors (ESFT) and one osteosarcoma cell line unrelated to ESFT as control of bone tumor cell line. Relative endogenous expression of each gene was normalized to actin (Average±SD, n=3).

Bioengineered Metastatic Tumors Using Mouse Models of Prostate Cancer

The predominant site of human prostate cancer metastasis is bone. Bone metastasis is the most frequent cause of death from prostate cancer. Genetically engineered mouse (GEM) models enable studies of metastasis in the native physiological milieu, and are suitable to model progression from tumorigenesis to metastasis. However, GEM models only rarely metastasize to bone, and fail to recapitulate the heterogeneity of human cancer phenotypes. In fact, a GEM model of fully penetrant metastatic prostate cancer displays metastases to many soft tissue sites but rarely if ever to bone. However, cells derived from this mouse model (i.e., NPK cells) readily form tumors when injected into the tibia.

The present disclosure combines generating mouse models of prostate cancer with tissue-engineering techniques, to evaluate prostate cancer metastasis in human bone context. The early metastasis tumor model can be evaluated by comparing to colonization of human or mouse prostate cancer cells injected through blood circulation into host mice that have been grafted with human or mouse bone. The advanced metastasis model can be evaluated by comparing to tumors formed by injecting human or mouse cancer cell aggregates directly into the grafted human or mouse bone. The host mice for these analyses can be non-obese diabetic/severe combined immunodeficient (NOD/SCID) mice engrafted with human bone.

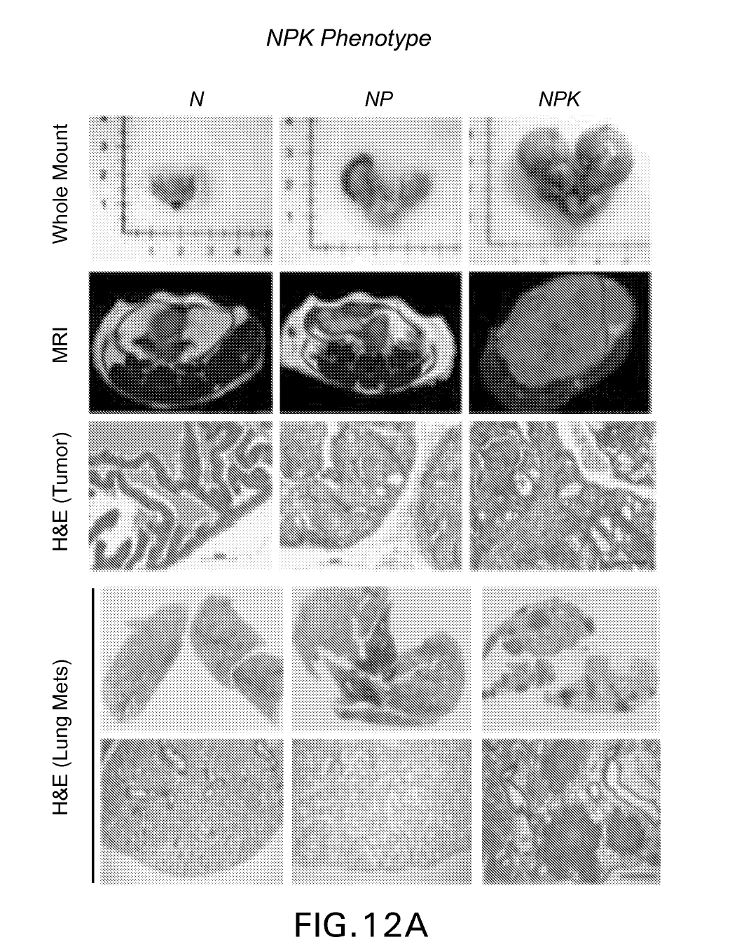

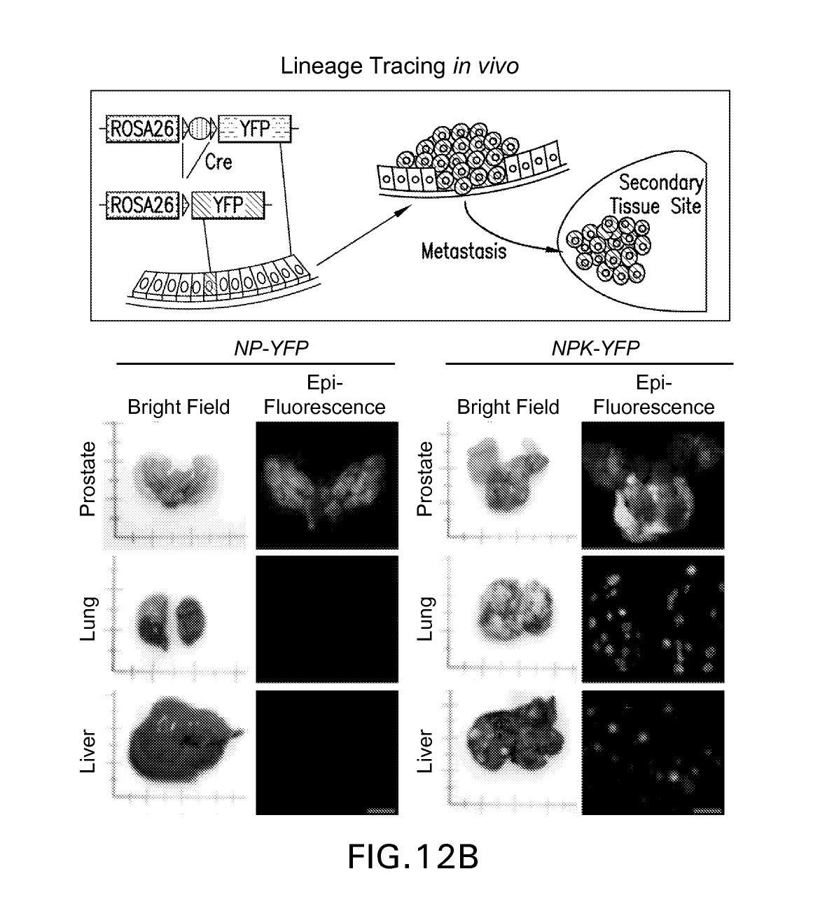

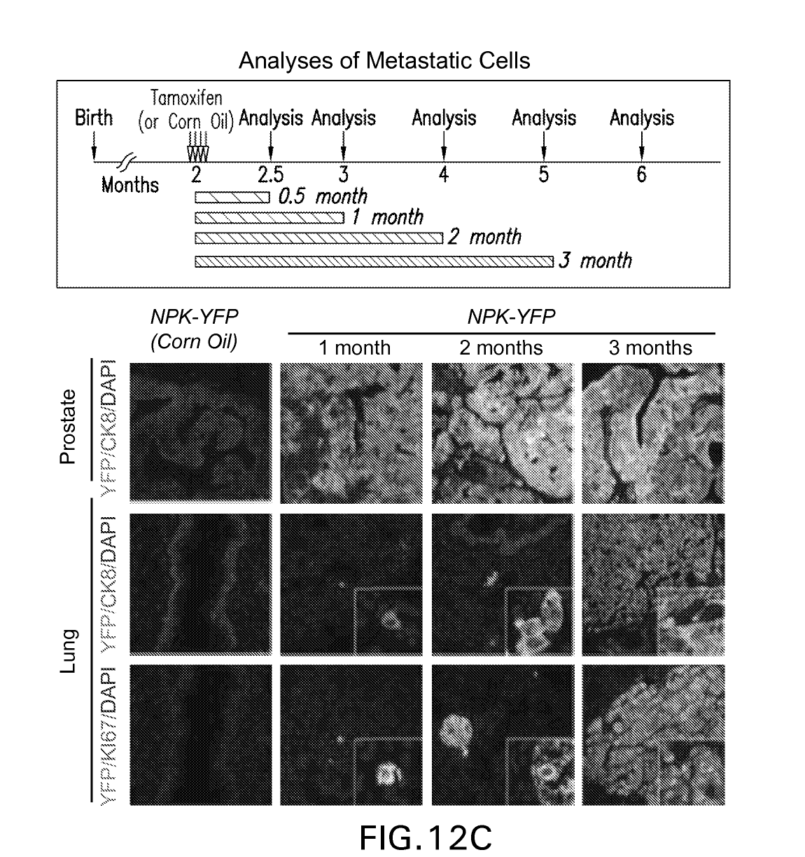

A series of GEM models are provided that display a range of prostate cancer phenotypes and share conserved molecular pathways deregulated in human prostate cancer and particularly activation of PI3-kinase and MAP kinase signaling pathways. In particular, while NP (Nkx3.1 CreERT2/+; Ptenflox/flox) tumors do not metastasize, NPK (Nkx3.1CreERT2/+; Ptenflox/flox; KrasLSL-G12D/+) tumors metastasize with nearly 100% penetrance to lymph nodes and soft tissues, most frequently to lungs and liver (FIGS. 12A-B), but not into the mouse bone. However, when implanted directly into the bone of host mice, the bone is rapidly colonized by the mouse tumor cells (FIG. 13D).

Lineage-tracing experiments using a Cre reporter allele R26R-YFP that indelibly marks prostate tumor cells, shows prominent YFP fluorescence in prostate tumors, lungs and livers from NPK mice that display metastases, but not in lungs or livers from NP mice that do not display metastases (FIG. 12B). Lineage-tracing is used to delineate the temporal and spatial relationship of tumors, disseminated cells, and metastases in the NPK mice, to observe a clear temporal delay in the appearance of metastasis which appear at 2-3 months relative to primary tumors which appear after only 1 month (FIG. 13C).

Metastasis Assay for Prostate Cancer Using the Bone-Engineered System

By using both human (PC3—highly metastatic and 22Rv1—non-metastatic) and mouse (NP-non metastatic, NPK-highly metastatic) prostate cancer cells, prostate cancer metastasis can be studied in a tissue- and species-specific manner, to determine whether the mouse bone provides the permissive microenvironment for prostate cancer metastasis as does human bone. These studies can be performed with both human and mouse prostate cancer cells. It is distinguishable whether preferential homing of human prostate cancer to bone (which cannot be readily recapitulated in mouse models) reflects a property of the primary tumor cells (human versus mouse) or whether tumor cells have a selective preference for human bone regardless of whether they are derived from mice or man.

To follow the cells in vivo the human PC3 and 22Rv1 cells are transduced with retroviral particles to stably express a dual luciferase-RFP reporter using a pMXs-IRES-Luc-RFP retroviral vector (Abate-Shen lab). Mouse NP and NPK cells are derived from mice already carrying a lineage tracing allele based on the expression of the YFP protein under the control of the R26r promoter. These cells are transduced to stably express a luciferase reporter by removing the RFP cassette. First, human pre-vascularized engineered bone (4×4 mm discs) is generated by sequential culture of hMSCs and HUVECs in bone scaffolds. After 4 weeks, engineered bone is implanted subcutaneously in male NOG/SCID mice for 10 days, a period that is sufficient to allow bone vascularization. Ten days post-implantation, 2.5×105 PC3 or NPK cells are injected into the tail vein with the luciferase-marked human or mouse prostate cancer cells, as above, and the mice are monitored twice a week for tumor formation in distant organs including the bone, using a Xenogen IVIS imaging system 15 minutes after intraperitoneal injection of 1.5 mg D-Luciferin. This model is compared to an early metastasis model. In separate animals, not implanted with human bone, human PC3 and mouse NPK cells transduced with luciferase reporter will be injected (105 cells per mouse) directly into the mouse tibia (FIG. 13C), to be compared with the advanced metastasis model. Second, 105 cells are implanted orthotopically into the mouse prostate and monitored over a period of 3 months for dissemination to distant organs, and into the implanted engineered bones (human and mouse). This assay provides the most stringent conditions for recapitulating almost entirely the initial steps of local invasion and extravasation.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A three-dimensional cancer model, the model comprising:
a decellularized bone scaffold; and
a plurality of cells arrayed on the scaffold.

2. The model of claim 1, wherein the plurality of cells comprises cancer cells.

3. The model of claim 2, wherein the cancer cells are selected from the group consisting of:
metastatic cancer cells, prostate cancer cells, and Ewing's sarcoma cells.

4. The model of claim 3, wherein the cancer cells comprise a plurality of spheroids.

5. The model of claim 1, wherein the bone scaffold comprises a plurality of perfusion channels.

6. The model of claim 1, wherein the plurality of cells comprises stem cells.

7. The model of claim 1, wherein the plurality of cells comprises osteoblasts.

8. The model of claim 1, wherein the plurality of cells comprises bone tissue cells.

9. The model of claim 1, wherein the plurality of cells comprises patient-derived cells.

10. The model of claim 1, wherein the scaffold is adapted for insertion in one well of a multiple well plate.

11. The model of claim 1, wherein the scaffold is adapted for insertion in one well of a 96-well plate.

12. The model of claim 1, wherein the scaffold is adapted for insertion in one well of a 24-well plate.

13. The model of claim 1, wherein the scaffold has an outer region, and inner region, and a core region.

14. The model of claim 13, wherein a first portion of the plurality of cells is arrayed in the outer region, a second portion of the plurality of cells is arrayed in the inner portion, and a third portion of the plurality of cells is arrayed in the core region, wherein the second portion is hypoxic and the third portion is necrotic.

15. A platform for modelling cancer, the platform comprising:
a decellularized bone scaffold;
an oxygen supply in gaseous communication with the bone scaffold;
a vasculature in fluid communication with the bone scaffold; and
a mechanical load coupled to the bone scaffold.

16. The platform of claim 15, wherein the mechanical load is adapted to apply a mechanical stress to the bone scaffold.

17. The platform of claim 15, wherein the vasculature comprises a nutrient supply.

18. A bioreactor comprising:
a decellularized bone scaffold;
an oxygen supply in gaseous communication with the bone scaffold;
a vasculature in fluid communication with the bone scaffold.

19. The bioreactor of claim 18, wherein the bioreactor is adapted to provide a biomimetic microenvironment to the scaffold.
